(19) United States
(12) Patent Application Publication
(10) Pub. No.: US 2026/0225116 A1
(43) Pub. Date: Aug. 6, 2026

(54) Spray Device and Method of Manufacturing the Same

(71) Applicant: Medspray B.V., Enschede (NL)

(72) Inventors: Wietze Nijdam, Enschede (NL); Henri Joseph Van Egmond, Enschede (NL)

(21) Appl. No.: 19/151,640

(22) PCT Filed: Feb. 5, 2024
(86) PCT No.: PCT/IB2024/051035

§ 371 Date: Jul. 29, 2025

(30) Foreign Application Priority Data
Feb. 3, 2023 (NL) 203-4082

(51) Int. Cl. B05B 1/08

(52) U.S. Cl. CPC B05B 1/086

(57) ABSTRACT

A spray device comprises a spray body with at least one cavity at a main surface and a membrane layer overlying said cavity. Said membrane layer comprises at least one spray opening communicating with said cavity and is arranged and configured to receive a pressurized liquid at a back side and to release at least one ray of liquid droplets at a front side thereof. Said cavity extends between an edge of said spray body and an opposite edge of said spray body. Said membrane layer comprises a reinforcement structure that extends from at least adjacent said first edge to at least adjacent said opposite edge over said cavity.

The present invention relates to a spray device, comprising a spray body comprising at least one cavity at a main surface and a membrane layer overlying said cavity, said membrane layer having a back side facing said cavity and an opposite front side, wherein said membrane layer comprises at least one spray opening between said back side and said front side communicating with said cavity and is arranged and configured to receive a pressurized liquid at said back side and to release at least one ray of liquid droplets at said front side.

A spray device of the above kind is for instance known from European patent EP 2.177.272.This known device comprises a spray nozzle that is built in a semiconductor body of silicon having at least one cavity emerging at a main surface of said body. Said main surface is covered by a ceramic membrane layer of silicon nitride that has at least one microscopic spray opening at the location of said cavity and in open communication with said cavity. A pressurized liquid is supplied through said cavity to said spray opening and break-up beyond said orifice into a droplet train of substantially identical spray droplets, having a size that resembles roughly between two and three times a diameter of said orifice. This phenomenon is generally known as Rayleigh breakup and is caused because the surface tension of the fluid is no longer capable of keeping a narrow ray of said liquid together as it passes beyond said orifice. The spray nozzle may have a number of such spray openings to deliver a uniform spray from said liquid without the need of a propellant.

Although Rayleigh breakup offers many benefits especially in terms of a droplet size distribution, it requires a substantial liquid pressure to force the liquid through the tiny spray opening(s) of the nozzle involved. This liquid pressure easily exceeds 1-2 MPa which put a significant stress on the brittle ceramic nitride membrane layer, with the risk of breakage. This is especially a concern with one or more spray openings at a relatively high pore density per cavity in terms of occupied surface by the spray opening(s) divided by the total cross sectional area of the cavity.

It is inter alia an object of the present invention to provide for a spray device and method of manufacturing the same in which said risk is significantly alleviated.

In order to attain said object, a spray device according to the present invention is characterized in that said cavity extends between an edge of said spray body and an opposite edge of said spray body, and in that said membrane layer comprises a reinforcement structure that extends from at least adjacent said first edge to at least adjacent said opposite edge over said cavity. The reinforcement structure that is added, according to the invention, to the ceramic layer helps to withstand the pressure that is exerted by the liquid during operation. Particularly the reinforcement structure counteracts a bending of the membrane layer as it stretches over a full dimension of the cavity from one edge to the opposite edge. Prior art spray devices having for instance more than two spray openings of 10 micron within a circular cavity of 50 micron diameter tend to be especially vulnerable to fracture at one of said edges where, hence, according to the invention the reinforcement is provided to avoid such breakage.

A particular embodiment of the spray device according to the invention is accordingly characterized in that said membrane layer comprises a plurality of spray openings between said back side and said front side communicating with said cavity, said spray openings being placed at an unprecedented spray opening density exceeding 8%, particularly exceeding 10% and more particularly exceeding 15%, in terms of occupied area by said spray openings divided by the total cross sectional surface of said cavity at said back side of said membrane layer. Having micron sized spray openings and an correspondingly sized cross sectional area of the cavity, the membrane layer in prior art devices tend to be considerably prone to rupture if the spray openings density would exceed such numbers. The reinforcement structure of the invention, however, provides sufficient additional strength to allow these numbers of micron sized or sub-micron sized openings in the membrane layer without adversely affecting the quality and reliability of the membrane layer.

In order not to interfere with the spray behaviour of the spray device, a preferred embodiment of the spray device according to the invention is characterized in that said membrane layer comprises said reinforcement structure at said back side. The frontal spray side of the membrane layer, hence, may remain unchanged to deliver a same spray pattern.

Although the reinforcement structure may be configured in many fashions, a particular embodiment of the spray device according to the invention is characterized in that said reinforcement structure comprises at least one rib extending between said first edge of said cavity and said opposite edge of said cavity. The one or more ribs, that are provided between both edges of the cavity, may extend all parallel to one or another or may cross each other to provide together some kind of waffle shaped reinforcement grid.

The one or more spray openings may then be placed in an inner space of such structure. Preferably the reinforcement structure is anchored in the wall of the cavity to provide additional strength and bending resistance. To that end, a further preferred embodiment of the spray device, according to the invention, is characterized in that said reinforcement structure extends into a wall of said cavity at at least one of said first edge and said opposite edge of said cavity, preferably at both of said first edge and said opposite edge of said cavity.

A still further preferred embodiment of the spray device according to the invention is characterized in that said reinforcement structure is formed integrally with said membrane layer. By integrating the reinforcement structure within the membrane layer a seamless transfer of additional rigidity and bending resistance is afforded to the membrane layer. As will become apparent from the below method according to the invention, the reinforcement structure may moreover be provided this way without requiring any additional deposition step during manufacturing.

The spray device may further benefit from the presence of the reinforcement structure to create a deflection of the spray ray that is released by said orifice. To that end a special embodiment of the spray device according to the invention is characterized in that said reinforcement structure is provided at said back side of said membrane layer, in that at least one spray opening of said at least one spray opening is provided in the vicinity of said reinforcement structure, a free length of said membrane layer between said spray opening and said reinforcement structure being smaller than a free length of said membrane layer at an opposite side of said spray opening away from said reinforcement structure.

It has been recognized that the reinforcement structure may modify the flow resistance that is experienced by said liquid as it passes from said cavity to said spray opening. Particularly, when said flow resistance is off-balance by positioning the spray opening closer to said reinforcement structure at one side than at another side of the opening, the difference in flow resistance happens to cause a deflection of said spray ray relative to a centre line of said spray opening in the direction of said one side. This effect may advantageously be used to create a modified spray pattern offered by said spray opening, notably a number of spray openings in community.

The spray device according to the invention is especially beneficial for creating a spray pattern of substantially identical, or at least precisely determined, micron sized droplets. To that end, the one or more spray openings may be created with high precision to transfer a same degree of precision to the droplets that are being generated. To that end a further preferred embodiment of the spray device according to the invention is characterized in that said spray body comprises a semiconductor body, particularly a silicon body, in that said membrane layer comprises a ceramic layer, particularly a silicon nitride layer, and in that said one or more spray openings have photo lithographically determined micron sized or sub-micron sized dimensions, typically between one or more micron and a few millimetres. The use of said materials allows the use of nowadays high definition semiconductor manufacturing techniques and tools for the production of an extremely precise spray nozzle to deliver an equally well defined spray pattern.

The present invention further offers a manufacturing process for manufacturing a spray device of the kind as described hereinbefore. To that end a method of manufacturing a spray device comprises the steps of:
providing a semiconductor body, particularly a silicon body, having a main surface;
providing at least one trench at said main surface in an exposed area of said semiconductor body;
oxidizing said exposed area and said at least one trench, forming an oxide layer on said exposed area, partially filling said at least one trench;
depositing a membrane layer on said oxide layer, said membrane layer filling said trench;
creating a spray opening in said membrane layer;
providing a cavity from a backside of said semiconductor body to expose said oxide layer at said exposed area; and
removing said oxide layer from within said cavity.

After said oxide layer is removed from with said cavity the portions of the membrane layer that filled the one or more trench will remain to form a reinforcement structure of said membrane layer at a back side of said layer.

A particular embodiment of the method according to the invention is characterized in that said exposed area of said semiconductor body is defined by an oxidation mask and in that said exposed area, comprising said at least one trench, is thermally oxidized masked by said oxidation mask, particularly in the presence of water, water vapour and/or steam. This oxidation process, generally referred to as LOCOS, creates a well defined edge to the cavity featuring a characteristic birds beak that provides a stress relief to the membrane layer.

Hereinafter, the invention will be described in further detail with reference to a specific embodiment and an accompanying drawing. In the drawing:

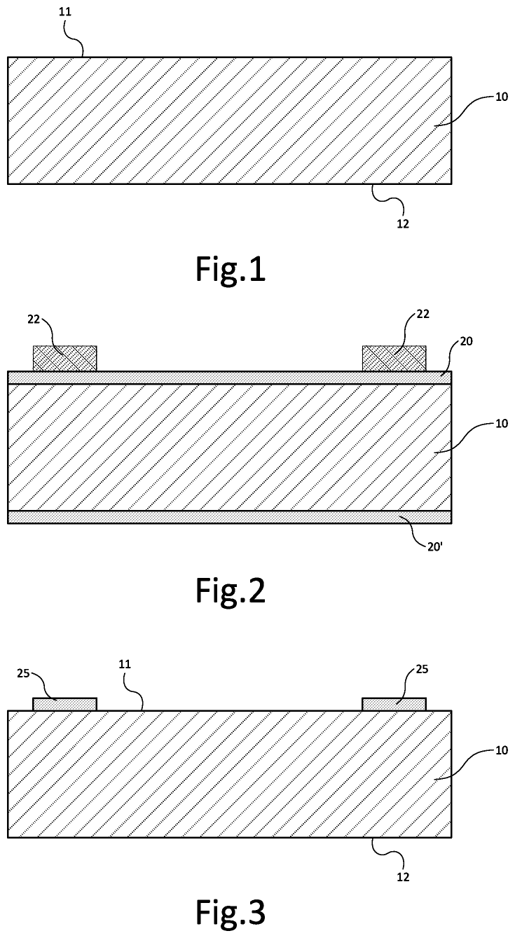

FIGS. 1-11 show in cross section consecutive process steps during an embodiment of a method according to the invention for manufacturing a spray device according to the invention;

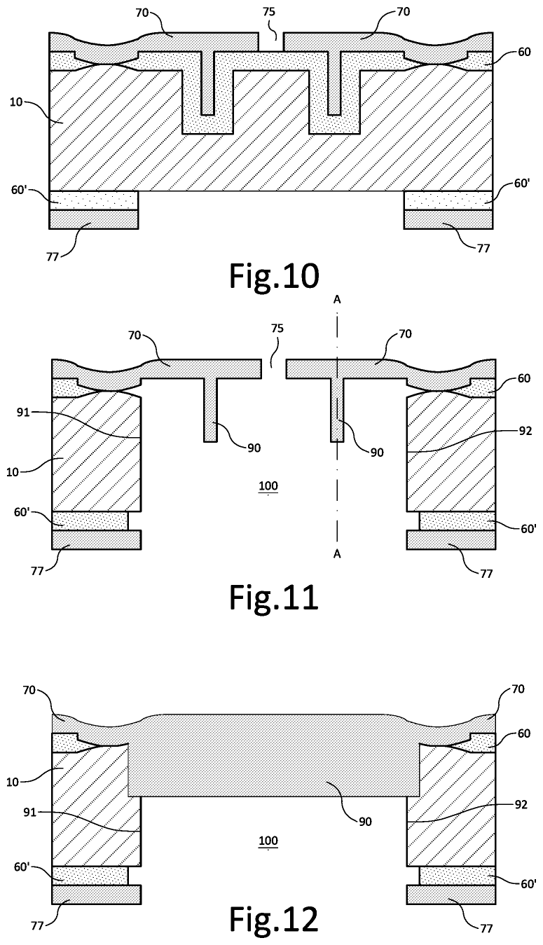

FIG. 12 shows a cross section perpendicular to that of FIG. 10 along the line A-A in FIG. 10; and

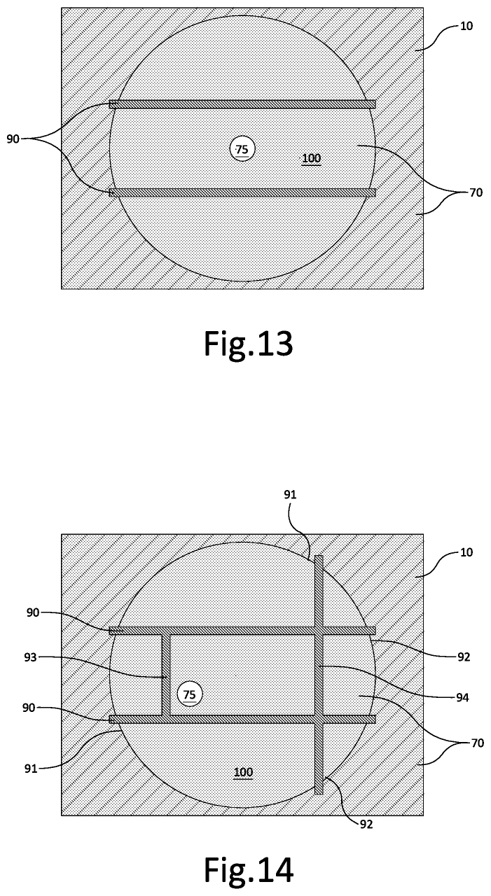

FIG. 13 shows a top view of the device of FIGS. 10 and 11.

It is noted that the figures are drawn purely schematically and not necessarily to a same scale. In particular, certain dimensions may have been exaggerated to a more or lesser extent to aid the clarity of any features. Similar parts are generally indicated by a same reference numeral throughout the figures.

For manufacturing a spray device according to the invention especially use can be made of existing semiconductor technology for creating a spray nozzle with a high degree of precision. FIG. 1 shows a starting process step of a method to produce a spray device according to the invention having a membrane layer that includes a reinforcement structure. To that end use is made of a silicon body 10 having a main surface 11 and a back side 12, see FIG. 1. The silicon body 10 typically has a thickness in the order of between 200 and 800 micron, particularly between 250 and 400 micron.

The silicon body 10 is covered by a silicon nitride layer 20,20′ by means of low pressure chemical vapour deposition. The deposited silicon nitride layer 20,20′ extends both at the main surface 11 as well as at the back side 12 of the silicon body 10, see FIG. 2. A suitable photo resist layer 22 is applied to the nitride layer 20 and is patterned using state of the art photo lithographic imaging and masking techniques. The resulting etch mask is used to etch the nitride layers 20,20′ at the areas that are left exposed, both at the main surface and the back to obtain an oxidation mask 25 as shown in FIG. 3.

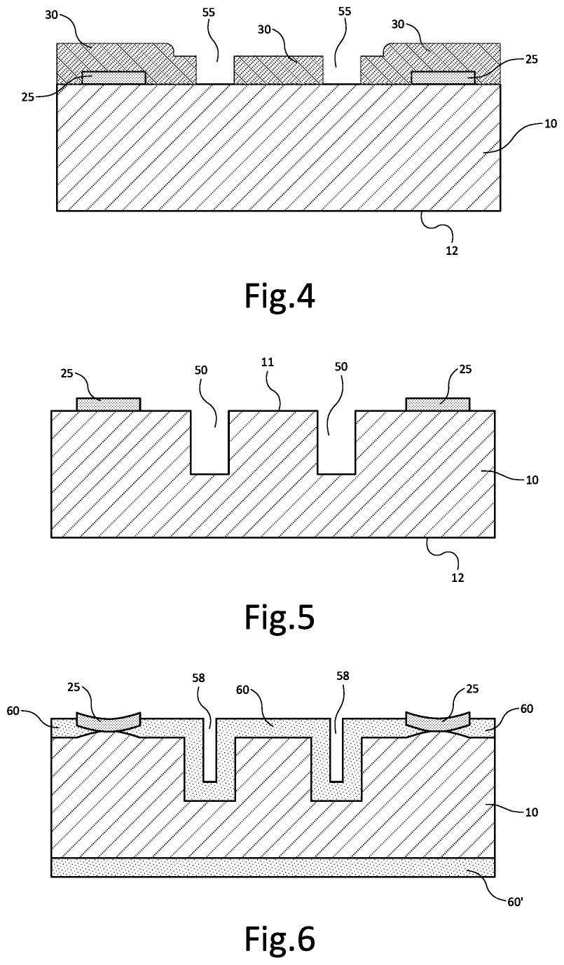

Next a photo resist layer 30 is deposited and is patterned using state of the art semiconductor photo-lithographic masking and developing techniques to create an etch mask that will protect the covered part of the silicon body 10 and the silicon nitride oxidation mask 25, see FIG. 4. The portions of the silicon body 10 that are left exposed are subjected to anisotropic reactive ion etching while the etch mask 30 protects the silicon surface. This creates a few steep trenches 50 at the main surface of the silicon body 10, having a width in the order of between 0.5 and 2,5 micron and a depth typically between 3 and 40 micron, preferably between 5 and 20 micron, depending on the reactive ion impact and duration, see FIG. 5.

The assembly is subsequently subjected to thermal oxidation to create a silicon oxide layer 60,60′ at the areas of the main surface 11 and back side 12 of the silicon body 10 that are left exposed by the oxidation mask 25, see FIG. 6. This process, generally referred to as LOCOS, creates a typical birds beak at the border of the oxidation mask 25 and only partly fills the trenches 50 to leave a gap 58 in the middle. Typically about 1 micron of silicon is consumed during this process such that the gap 58 will remain of between 0.1 and 2 micron wide, particularly of about 0.5 micron wide.

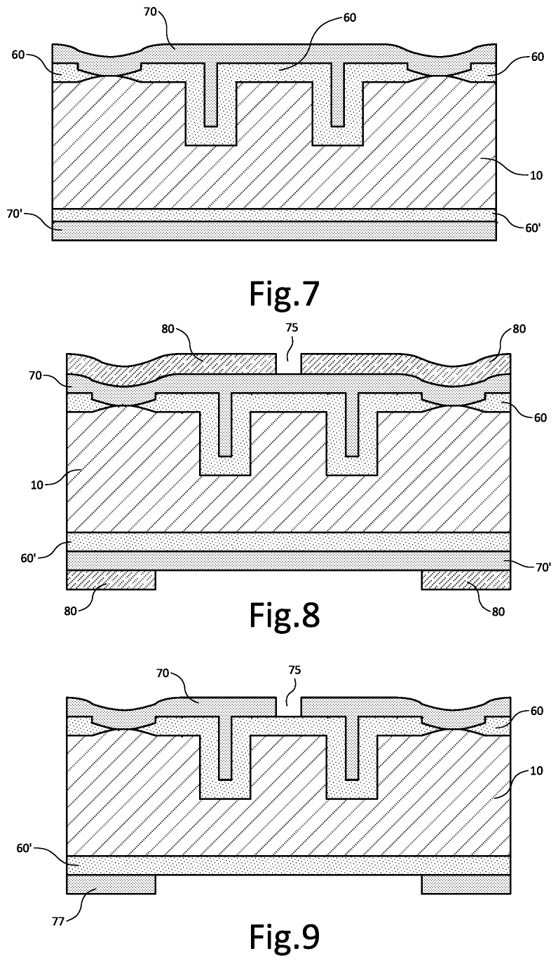

After removal of the oxidation mask 25, a conformal silicon nitride layer 70,70′ is deposited on the entire surface using low pressure chemical vapour deposition. This layer 70,70′ is in the order of between 0,3 and 5 micron thick, particularly about 1 micron, and will serve as membrane layer in the final product. At the same time the deposited silicon nitride completely fills the gaps 58 that were left of the trenches 50, see FIG. 7.

Using a further patterned photo resist lacquer 80, one or more spray openings 75 are defined in the silicon nitride layer 70 at the main surface of the silicon body 10 and concurrently an etch mask 77 is defined in the silicon nitride layer 70′ at the back side of the body 10, see FIG. 8. Next the spray openings 75 are created by etching, while concurrently said etch mask 77 is formed out of the nitride layer 70′ at the back side, see FIG. 9.

Using said etch mask 77, the exposed part of the oxide layer 60′ is removed from the back side of the silicon body 10, see FIG. 10. Subsequently, the silicon body 10 is etched throughout its thickness from the back side, using the patterned oxide back layer 60′ as an etch mask, until the top oxide layer 60 is reached, see FIG. 11. This creates a cavity 100 within the silicon body 10 having a width that is typically of the order of between 30 and 200 micron, and preferably between 50 and 100 micron. In this example, the cavity 100 has a round cross section with a diameter of about 50 micron.

Finally the silicon oxide layer 60 is removed by selective etching to expose the silicon nitride membrane layer 70 from within the cavity 100. At the same time the oxide layer 60 is removed from the portions 90 of the nitride layer 70 that filled the gaps 55 within the trenches 50. These nitride portions 90 form a reinforcement structure integral with the nitride membrane layer 70.

The resulting device is shown in FIG. 12 in a traverse cross section along the line A-A of FIG. 11 as well as in FIG. 13 in top view. As shown in the figure the reinforcement ribs 90 extend from one edge 91 to and opposite edge 92 of the cavity 100 and even a little into the silicon structure 10 surrounding the island defined by the cavity 100 to provide an anchoring of the reinforcement structure 90 into the silicon body 10.

The one or more reinforcement ribs 90, that are provided between both edges of the cavity, may extend all parallel to one or another, as shown in FIGS. 12 and 13, or may alternative cross further reinforcement ribs 93,94, traverse or inclined, to form together any kind of waffle-like reinforcement grid as shown in FIG. 14. The reinforcement ribs 90,93,94 may all extend into the silicon boundary of the cavity 100 or one or more reinforcement ribs 93 may be shorter as shown in the figure. One or more spray openings 75 may be placed in an inner space of such structure either centrally or away from the centre.

Although the invention has been described herein before with reference to merely a limited number of explanatory embodiments, it should be understood that the invention is by no means limited to those examples. On the contrary many more variations and embodiments are feasible to a skilled person within the framework of the present invention without requiring him or her to exercise any inventive skill. Particularly the reinforced membrane structure and device according to the invention might as well be applied and implemented mutatis mutandis to serve as a micro sieve or filter for fluids, particularly liquids and gasses to filter. Such filter or sieve may be used to remove micron sized or sub-micron sized contaminating particles that are present in the fluid or to harvest micron sized or sub-micron sized entities from within the fluid.

1. A spray device, comprising a spray body comprising at least one cavity at a main surface and a membrane layer overlying said cavity, said membrane layer having a back side facing said cavity and an opposite front side, wherein said membrane layer comprises at least one spray opening between said back side and said front side communicating with said cavity and is arranged and configured to receive a pressurized liquid at said back side and to release at least one ray of liquid droplets at said front side, wherein said cavity extends between an edge of said spray body and an opposite edge of said spray body, and in wherein said membrane layer comprises a reinforcement structure that extends from at least adjacent said first edge to at least adjacent said opposite edge over said cavity.

2. The spray device according to claim 1, wherein said membrane layer comprises said reinforcement structure at said back side.

3. The spray device according to claim 1, wherein said reinforcement structure comprises at least one rib extending between said first edge of said cavity and said opposite edge of said cavity.

4. The spray device according to claim 1, wherein said reinforcement structure extends into a wall of said cavity at at least one of said first edge and said opposite edge of said cavity, preferably at both of said first edge and said opposite edge of said cavity.

5. The spray device according to claim 1, wherein said reinforcement structure is formed integrally with said membrane layer.

6. The spray device according to claim 1, wherein said reinforcement structure is provided at said back side of said membrane layer, wherein at least one spray opening of said at least one spray opening is provided in the vicinity of said reinforcement structure, a free length of said membrane layer between said spray opening and said reinforcement structure being smaller than a free length of said membrane layer at an opposite side of said spray opening away from said reinforcement structure.

7. The spray device according to claim 1, wherein said spray body comprises a semiconductor body, wherein said membrane layer comprises a ceramic layer, particularly a silicon nitride layer, and wherein said one or more spray openings have photo lithographically determined micron sized or sub-micron sized dimensions, typically between one or more micron and a few millimetres.

8. The spray device according to claim 1, wherein said membrane layer comprises a plurality of spray openings between said back side and said front side communicating with said cavity, said spray openings being placed at a density exceeding 8%, in terms of occupied area by said spray openings divided by the total cross sectional surface of said cavity at said back side of said membrane layer.

9. A method of manufacturing a spray device comprising the steps of:
providing a semiconductor body, having a main surface;
providing at least one trench at said main surface in an exposed area of said semiconductor body;
oxidizing said exposed area and said at least one trench, forming an oxide layer on said exposed area, partially filling said at least one trench;
depositing a membrane layer on said oxide layer, said membrane layer filling said trench;
creating a spray opening in said membrane layer;
providing a cavity from a backside of said semiconductor body to expose said oxide layer at said exposed area; and
removing said oxide layer from within said cavity.

10. The method according to claim 9, wherein said exposed area of said semiconductor body is defined by an oxidation mask and in that said exposed area, comprising said at least one trench, is thermally oxidized masked by said oxidation mask.

11. The spray device according to claim 7, wherein said semiconductor body comprises a silicon body and wherein said ceramic layer comprises a silicon nitride layer.

12. The spray device according to claim 8, wherein said spray openings being placed at a density exceeding 10% in terms of occupied area by said spray openings divided by the total cross sectional surface of said cavity at said back side of said membrane layer.

13. The spray device according to claim 12, wherein said spray openings being placed at a density exceeding 15% in terms of occupied area by said spray openings divided by the total cross sectional surface of said cavity at said back side of said membrane layer.

14. The method according to claim 9, wherein said semiconductor body comprises a silicon body.

15. The method according to claim 14, wherein said at least one trench, is thermally oxidized masked by said oxidation mask in the presence of water, water vapour and/or steam.

16. The spray device according to claim 2, wherein said reinforcement structure comprises at least one rib extending between said first edge of said cavity and said opposite edge of said cavity.

17. The spray device according to claim 2 wherein said reinforcement structure extends into a wall of said cavity at least one of said first edge and said opposite edge of said cavity, preferably at both of said first edge and said opposite edge of said cavity.

18. The spray device according to claim 3 wherein said reinforcement structure extends into a wall of said cavity at least one of said first edge and said opposite edge of said cavity, preferably at both of said first edge and said opposite edge of said cavity.

19. device according to claim 2, wherein said reinforcement structure is formed integrally with said membrane layer.

20. device according to claim 3, wherein said reinforcement structure is formed integrally with said membrane layer.
